(19) United States
(12) Patent Application Publication
(10) Pub. No.: US 2022/0096242 A1
(43) Pub. Date: Mar. 31, 2022

(54) MTS MEDIAL TIBIAL PLATEAU PATCH, MODULAR MTS MEDIAL TIBIAL PLATEAU PATCH AND MINIMALLY INVASIVE REPLACEMENT METHOD THEREFOR

(71) Applicants: Yan WANG, Beijing (CN); Wei CHAI, Beijing (CN)

(72) Inventors: Yan WANG, Beijing (CN); Wei CHAI, Beijing (CN)

(21) Appl. No.: 17/427,113

(22) PCT Filed: Apr. 15, 2019
(86) PCT No.: PCT/CN2019/082730

§ 371 Date: Jul. 30, 2021

(30) Foreign Application Priority Data
Mar. 11, 2019 (CN) 201910180639.5

(51) Int. Cl. A61F 2/38; A61F 2/30; A61F 2/46

(52) U.S. Cl. CPC A61F 2/389; A61F 2/30942; A61F 2/461; A61F 2002/30827; A61F 2002/4635; A61F 2002/30891; A61F 2002/30884; A61F 2002/30836; A61F 2002/30823; A61F 2002/30131

(57) ABSTRACT

An MTS medial tibial plateau patch, a modular MTS medial tibial plateau patch, and a minimally invasive replacement method thereof are disclosed. The MTS medial tibial plateau patch includes an articular surface provided with an arc-shaped recessed area and a bottom surface provided with 3 stand columns distributed triangularly, or two longitudinal keels having non-parallel distribution or two triangular keel wings, and the arc-shaped recessed area is well matched and in a sliding fit with a femoral condyle without an increase in impact in a joint, thus guaranteeing a stability of a prosthesis, realizing a concept of a minimally invasive surgery, and reducing damage to a normal tissue. The modular MTS medial tibial plateau patch includes a bone-trabecula metal tibial support and a liner made of VE high cross-linked polyethylene.

### CROSS REFERENCE TO THE RELATED APPLICATIONS

This application is the national stage entry of International Application No. PCT/CN2019/082730, filed on Apr. 15, 2019, which is based upon and claims priority to Chinese Patent Application No. 201910180639.5, filed on Mar. 11, 2019, the entire contents of which are incorporated herein by reference.

### TECHNICAL FIELD

The present invention relates to the field of artificial knee joint replacement, and particularly to an MTS medial tibial plateau patch, a modular MTS medial tibial plateau patch, and a minimally invasive replacement method thereof, which are applied to minimally invasive surgery replacement of patients with obvious wear of tibial articular surfaces, cartilage loss, or tibial deformity.

### BACKGROUND

With the advent of the aging society of China and the rapid development of medical and health services, the average life span is prolonged gradually, and arthritis, as degeneration, becomes a major medical problem which troubles the middle-aged and elderly people in today's society. The incidence of arthritis is particularly high in the knee joint which is a major weight-bearing joint. In addition, the obesity problem which is increasingly serious and sports injuries caused by various factors play a role in promoting the occurrence and development of knee osteoarthritis. The affected knee joint surface and cartilage are damaged, and obvious wear and loss of the surface cause pains and a limitation on the activity range of the knee joint, seriously influencing the daily life and work of a patient.

Current surgical protocols adopted at home and abroad include total knee arthroplasty (TKA), unicompartmental knee arthroplasty (UKA), and high tibial osteotomy (HTO).

The existing surgical protocols have the following disadvantages. 1. In the total knee arthroplasty, the medial and lateral compartments or/and the patellofemoral compartment as well as the cruciate ligaments are resected completely or partially, resulting in large surgical injuries; the total knee arthroplasty is undoubtedly impractical or inelegant for a patient only with single-compartment arthritis and sacrifices normal articular surfaces and ligaments, resulting in poor proprioception and a dissatisfaction rate after the total knee arthroplasty up to 20%. Once the arthroplasty has complications, such as infection, looseness, wear, dislocation, or the like, a revision surgery is a destructive surgery, has great difficulty, requires a special prosthesis, and is quite expensive. 2. In the unicompartmental knee arthroplasty, only the unilateral compartment articular surfaces of the tibia and the femur are resected, and a tibial prosthesis, a femoral prosthesis, and a liner prosthesis are implanted, resulting in good proprioception; however, components of this arthroplasty have limited service lives, the tibial plateau is loose, a liner has a high dislocation incidence, excessive bones are resected, and when being the total knee arthroplasty, a revision surgery has great difficulty and requires an extra accessory, such as a block, or the like, for fixation, which limits popularization of the unicompartmental knee arthroplasty. 3. The high tibial osteotomy is only effective for a gonitis patient with tibial deformity and requires an extra steel plate for fixation, resulting in a high cost; activities are required to be restricted after the high tibial osteotomy, which does not conform to the idea and demand of quick recovery of orthopedics patients. Researches show that a patient after the osteotomy has an altered anatomical position during the arthroplasty, and the functional effect is poorer than the functional effect of primary arthroplasty on a patient.

### SUMMARY

An object of the present invention is to provide an MTS medial tibial plateau patch, a modular MTS medial tibial plateau patch, and a minimally invasive replacement method thereof, which are used for solving the problems of large surgical injuries, high difficulty, short service lives, looseness of the tibial plateau, a high dislocation incidence of a liner, excessive resected bones, or the like, in the prior art.

In order to achieve the above-mentioned object, the present invention provides the following technical solution.

An MTS medial tibial plateau patch includes an articular surface provided with an arc-shaped recessed area and a bottom surface provided with 3 stand columns which are distributed triangularly or 2 longitudinal keels having non-parallel distribution or 2 triangular keel wings.

Each stand column and the bottom surface have an included angle of 20-40 degrees.

Each stand column is provided with 3-4 arc-shaped grooves which are distributed evenly.

Each longitudinal keel has a taper of 3-7 degrees.

Each longitudinal keel is provided with 3-5 convex ridges which are distributed uniformly.

Each triangular keel wing is provided with 2-4 anti-rotation arc grooves. The bottom surface takes the form of one of a surface with grid-shaped concave bone cement grooves, a surface with a plurality of dovetail-shaped bone cement grooves which are distributed longitudinally, and a polished surface without grooves.

The MTS medial tibial plateau patch has a D-shaped cross section and is bionically designed according to the anatomical form of the tibial plateau of the knee joint of a Chinese person, and prostheses have models 1-6 with different sizes.

The MTS medial tibial plateau patch further includes a plurality of tibial plateau patches with different thicknesses of 3-14 mm.

The articular surface has an equal thickness in a front-rear direction.

The MTS medial tibial plateau patch is made of VE high cross-linked polyethylene.

A modular MTS medial tibial plateau patch includes a tibial support and a liner, wherein the tibial support includes an upper surface provided with a conical protrusion and a bottom surface provided with 3-4 stand columns which are distributed triangularly or 2-3 longitudinal keels having non-parallel distribution; and the liner includes an articular surface provided with an arc-shaped recessed area and a lower surface provided with a conical recess;

the tibial support and the liner are locked by the conical protrusion of the tibial support and the conical recess of the liner.

The bottom surface of the tibial support is provided with a bone-trabecula porous structure layer with a thickness of 1-5 mm.

The tibial support is made of medical titanium alloy or cobalt chromium molybdenum alloy or polyether ether ketone (PEEK) or derivatives thereof, and the liner is made of VE high cross-linked polyethylene.

A minimally invasive replacement method for replacing the MTS medial tibial plateau patch or the modular MTS medial tibial plateau patch includes: exposing the tibial medial or lateral plateau of the affected limb of a patient with a minimally invasive surgery method with a small incision of a parapatellar approach; resecting bone tissue of the affected tibia according to coronal inversion and eversion angles as well as a sagittal retroversion angle planned before a surgery; and implanting the MTS medial tibial plateau patch onto the tibia after resection, wherein the integrated MTS medial tibial plateau patch is fixed to the tibia after resection by cement, and the modular MTS medial tibial plateau patch is biologically fixed to the tibia after resection.

The above-mentioned technical solution of the present invention has the following beneficial effects.

In the above-mentioned solution, the MTS medial tibial plateau patch and the modular MTS medial tibial plateau patch according to the present invention have simple structures and good form similarity and accord with anatomical features of Chinese people; the MTS medial tibial plateau patch and the modular MTS medial tibial plateau patch are convenient to implant, instruments are simple, the surgery is minimally invasive, important soft tissue is not touched, the patient recovers rapidly after the surgery, and outpatient surgery treatment may be expected to achieve hospital discharge on the same day; the MTS medial tibial plateau patch is fixed firmly and has strong wear resistance and high biocompatibility; for the minimally invasive replacement method of the MTS medial tibial plateau patch or the modular MTS medial tibial plateau patch, the quantity of resected bones is small, a revision surgery is simple, and the difficulty of total knee arthroplasty is not increased; and the MTS medial tibial plateau patch may reduce metal ion toxicity and anaphylactic reaction and does not affect nuclear magnetic resonance imaging.

### BRIEF DESCRIPTION OF THE DRAWINGS

FIG. 1 is a first perspective structural diagram of an MTS medial tibial plateau patch according to the present invention;

FIG. 2 is a second perspective structural diagram of the MTS medial tibial plateau patch according to the present invention;

FIG. 3 is a sagittal view of the first structural diagram of the MTS medial tibial plateau patch according to the present invention;

FIG. 4 is a third perspective structural diagram of the MTS medial tibial plateau patch according to the present invention;

FIG. 5A is a sagittal view of the third structural diagram of the MTS medial tibial plateau patch according to the present invention;

FIG. 5B is a partially enlarged view showing the portion A in FIG. 5A;

FIG. 6 is a fourth perspective structural diagram of the MTS medial tibial plateau patch according to the present invention;

FIG. 7 is a sagittal view of the fourth structural diagram of the MTS medial tibial plateau patch according to the present invention;

FIG. 8 is a perspective structural diagram of a modular MTS medial tibial plateau patch according to the present invention;

FIG. 9 is an exploded perspective view of the modular MTS medial tibial plateau patch according to the present invention;

FIG. 10A is a sagittal sectional view of the modular MTS medial tibial plateau patch according to the present invention;

FIG. 10B is a partially enlarged view showing the portion B in FIG. 10A;

FIG. 10C is a partially enlarged view showing the portion C in FIG. 10A;

FIG. 11 is an effect diagram before replacement of the MTS medial tibial plateau patch according to the present invention; and

FIG. 12 is an effect diagram after the replacement of the MTS medial tibial plateau patch according to the present invention.

### REFERENCE NUMERALS

1. bottom surface; 2. concave bone cement groove; 3. stand column; 4. longitudinal keel; 5. liner; 6. protrusion; 7. recess; 8. tibial support; 9. triangular keel wing; 10. dovetail-shaped bone cement groove; 11. cylindrical keel wing.

### DETAILED DESCRIPTION OF THE EMBODIMENTS

To make the technical problems to be solved, technical solutions and advantages of the present invention more apparent, the following detailed description is given with reference to the accompanying drawings and embodiments.

The present invention provides an MTS medial tibial plateau patch, a modular MTS medial tibial plateau patch, and a minimally invasive replacement method thereof, which are used for solving the problems of large surgical injuries, high difficulty, short service lives, looseness of the tibial plateau, a high dislocation incidence of a liner, and excessive resected bones in the prior art.

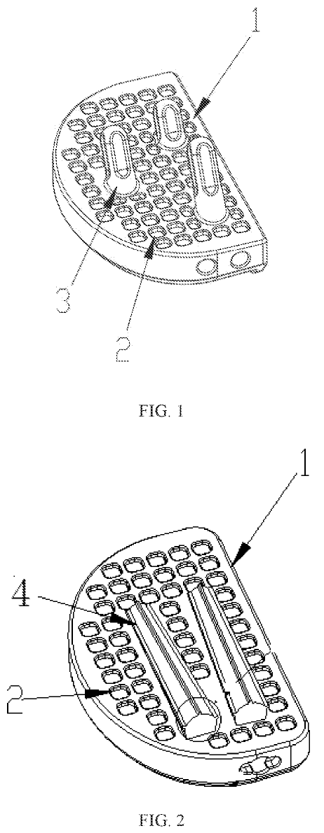

FIG. 1 shows an MTS medial tibial plateau patch according to a first embodiment of the present invention; the MTS medial tibial plateau patch includes an articular surface provided with an arc-shaped recessed area and a bottom surface 1 provided with 3-4 stand columns 3, preferably 3 stand columns 3, which are distributed triangularly, and the arc-shaped recessed area is well matched and in sliding fit with the femoral condyle without an increase in impact in the joint.

FIG. 2 shows an MTS medial tibial plateau patch according to a second embodiment of the present invention; the MTS medial tibial plateau patch includes an articular surface provided with an arc-shaped recessed area and a bottom surface 1 provided with 2-3 longitudinal keels 4, preferably 2 longitudinal keels 4, which have non-parallel distribution, and the arc-shaped recessed area is well matched and in sliding fit with the femoral condyle without an increase in impact in the joint.

FIG. 4 shows an MTS medial tibial plateau patch according to a third embodiment of the present invention; the MTS medial tibial plateau patch includes an articular surface provided with an arc-shaped recessed area and a bottom surface 1 provided with 2-3 triangular keel wings 9, preferably 2 triangular keel wings 9 in this embodiment, the arc-shaped recessed area is well matched and in sliding fit with the femoral condyle without an increase in impact in the joint, and the triangular keel wings 9 are distributed longitudinally and each provided with 2-4 anti-rotation arc grooves.

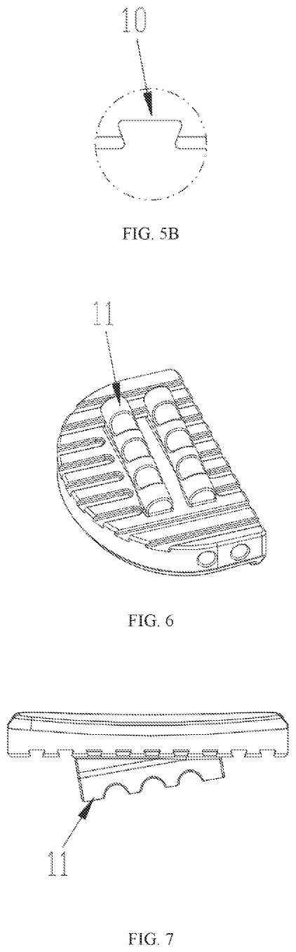

FIG. 6 shows an MTS medial tibial plateau patch according to a fourth embodiment of the present invention; the MTS medial tibial plateau patch includes an articular surface provided with an arc-shaped recessed area and a bottom surface 1 provided with 2-3 keel wings 11, preferably 2 cylindrical keel wings 11 in this embodiment, the arc-shaped recessed area is well matched and in sliding fit with the femoral condyle without an increase in impact in the joint, and the keel wings 11 are distributed longitudinally, have cylindrical sections and are each provided with 2-4 anti-rotation arc grooves.

The bottom surface takes the form of one of a surface with grid-shaped concave bone cement grooves, a surface with a plurality of dovetail-shaped bone cement grooves which are distributed longitudinally, and a polished surface without grooves.

The MTS medial tibial plateau patch according to the present invention has a thickness of 3-14 mm and may correct the deformity of the force line of the coronal plane within 0-15 degrees and recover the original tension of the joint ligament and the force line of the joint, so as to relieve pains. The MTS medial tibial plateau patch is made of VE high cross-linked polyethylene, thus reducing metal ion toxicity and anaphylactic reaction without influences on nuclear magnetic resonance imaging. The VE high cross-linked polyethylene has wear resistance which is extremely high, and compared with an existing common ultrahigh molecular weight polyethylene material, the VE high cross-linked polyethylene has good wear resistance, high bending strength, and high fatigue resistance, and has an elastic modulus closer to the elastic modulus of the cartilage of the articular surface of a human body, such that the articular surface of the MTS medial tibial plateau patch is better fitted with the articular surface of the human body. The MTS medial tibial plateau patch according to the present invention is fixed by cement and may be combined with free radicals after implanted, thus reducing the oxidation rate of the material.

A tibial support is made of medical titanium alloy or cobalt chromium molybdenum alloy or polyether ether ketone (PEEK) or derivatives thereof, and the liner is made of VE high cross-linked polyethylene.

The MTS medial tibial plateau patch according to the present invention may be set to have different thicknesses and different sizes according to shapes of human bones and has models 1-6, a front-back diameter and a left-right diameter achieve good matching effects, so as to correct different degrees of deformity of the force line of a patient, and the patient using the MTS medial tibial plateau patch according to the present invention has kinematic mechanical characteristics closer to the kinematic mechanical characteristics by a natural joint.

The articular surface of the MTS medial tibial plateau patch according to the present invention has an equal thickness in a front-rear direction, and an osteotomy angle is determined according to original retroversion of the joint to realize a normal backward rolling action of the joint in flexion and extension activities and recover the original eccentricity of the joint.

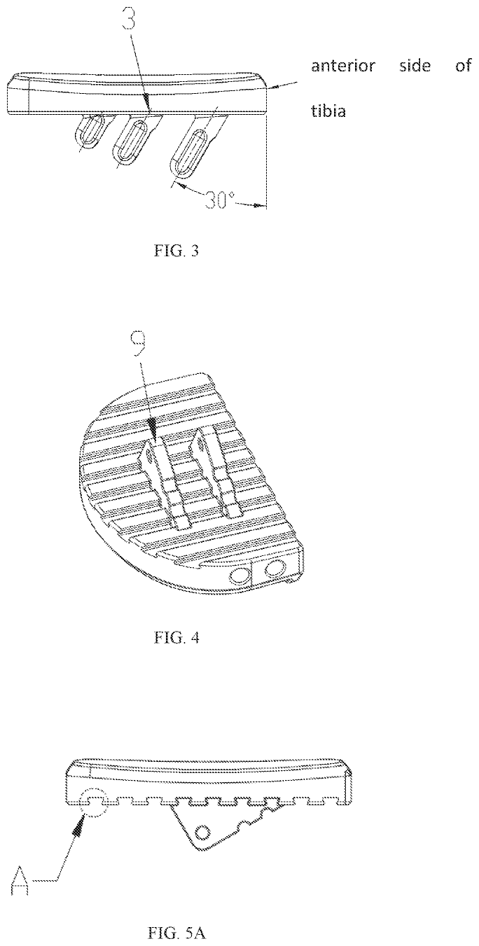

As shown in FIG. 3, the articular surface in the present invention is designed according to the shape and size of the meniscus of the knee joint of the human body, the MTS medial tibial plateau patch has a D-shaped cross section and is bionically designed according to the anatomical form of the tibial plateau of the knee joint of a Chinese person, and prostheses have models 1-6 with different sizes. An arc-shaped recess with small and flat radian is provided at the sagittal plane of the MTS medial tibial plateau patch.

The MTS medial tibial plateau patch further includes a plurality of tibial plateau patches with different thicknesses of 3-14 mm.

The articular surface has an equal thickness in the front-rear direction.

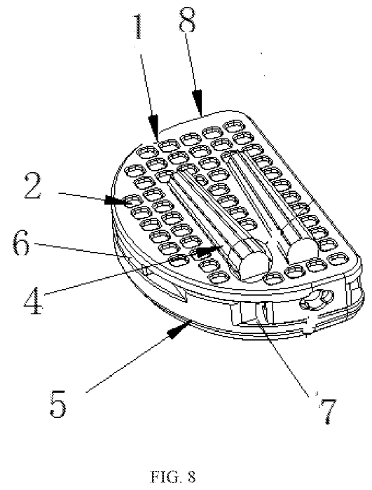

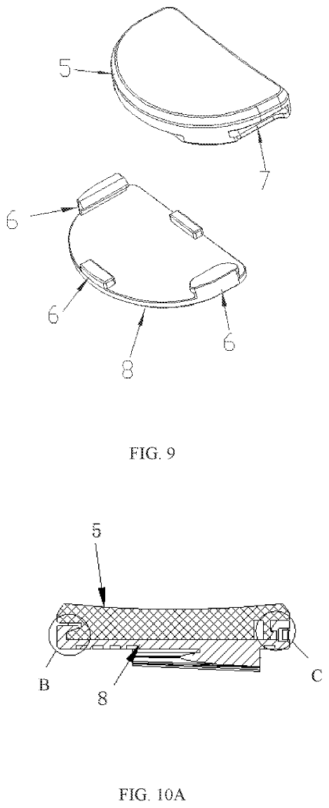

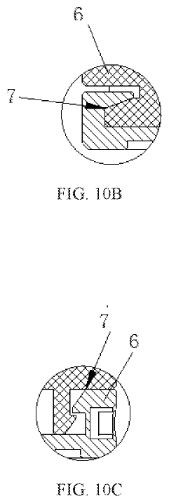

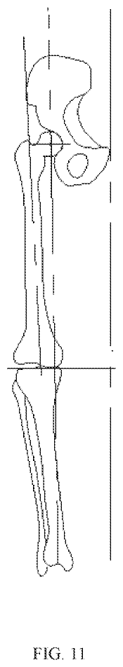

As shown in FIGS. 8-9, the embodiment of the present invention provides a modular MTS medial tibial plateau patch including a tibial support 8 and a liner 5, wherein the tibial support 8 includes an upper surface provided with a conical protrusion 6 and a bottom surface 1 provided with 3-4 stand columns which are distributed triangularly or 2-3 longitudinal keels having non-parallel distribution; and the liner includes an articular surface provided with an arc-shaped recessed area and a lower surface provided with a conical recess 7. As shown in FIGS. 10A-10C, the tibial support 8 and the liner 5 are locked by the conical protrusion 6 of the tibial support 8 and the conical recess 7 of the liner 5, thereby effectively preventing the modular tibial patch from loosening and guaranteeing the reliability of the combination of the patch and the joint. The lower surface of the liner 5 is matched with the upper surface of a plateau of the tibial support 8.

The bottom surface of the tibial support 8 is provided with a bone-trabecula porous structure layer with a thickness of 2-3 mm. The liner 5 has a thickness of 4-12 mm and may correct the deformity of the coronal plane within 0-15 degrees.

The modular MTS medial tibial plateau patch includes the bone-trabecula metal tibial support 8 with the best bone ingrowth performance currently and the liner 5 made of VE high cross-linked polyethylene, has an elastic modulus and porosity close to the elastic modulus and porosity of normal bone tissue, and is fixed biologically; the liner 5 is made of the VE high cross-linked polyethylene with wear resistance which is extremely high, and a prosthesis made of such a material may be combined with free radicals after implanted to reduce the oxidation rate of the material, and has vibration absorption and buffering effects, thus avoiding the stress shielding problem after a traditional surgery.

As shown in FIGS. 1 and 3, the stand columns 3 in the present invention are optimally designed according to stress conditions, thus facilitating stress decomposition and achieving excellent positioning and fixing effects. Each stand column 3 and the bottom surface 1 have an included angle of 28-31 degrees, preferably 30 degrees. Each stand column 3 is provided with three arc-shaped grooves which are distributed evenly, and the arc-shaped grooves may enhance the stability of the MTS medial tibial plateau patch, and meanwhile facilitate a mounting process of the MTS medial tibial plateau patch, reduce damage to normal tissue, and realize the concept of a minimally invasive surgery.

As shown in FIG. 2, the longitudinal keels 4 in the present invention are optimally designed according to stress conditions, thus facilitating stress decomposition and achieving excellent positioning and fixing effects. Each longitudinal keel 4 has a taper of 3-4 degrees. Each longitudinal keel 4 is provided with 3-5, preferably 3, convex ridges which are distributed uniformly. The longitudinal keels 4 may enhance the stability of the MTS medial tibial plateau patch, and meanwhile facilitate the mounting process of the MTS medial tibial plateau patch, reduce damage to normal tissue, and realize the concept of the minimally invasive surgery.

As shown in FIGS. 1 and 2, the bottom surface 1 in the present invention is further provided with grid-shaped projections 2 which facilitate fixation of the MTS medial tibial plateau patch and prevent the MTS medial tibial plateau patch from loosening or falling off.

As shown in FIGS. 4-7, the bottom surface 1 in the present invention is provided with a plurality of dovetail-shaped bone cement grooves 10 which are distributed longitudinally, and the dovetail-shaped bone cement grooves facilitate fixation of the MTS medial tibial plateau patch and prevent the MTS medial tibial plateau patch from loosening or falling off or moving forward.

An embodiment of the present invention provides a minimally invasive replacement method for replacing the MTS medial tibial plateau patch or the modular MTS medial tibial plateau patch, including: exposing the tibial medial or lateral plateau of the affected limb of a patient with a minimally invasive surgery method with a small incision of a parapatellar approach; resecting bone tissue of the affected tibia according to coronal inversion and eversion angles as well as a sagittal retroversion angle planned before a surgery; and implanting the MTS medial tibial plateau patch or the modular MTS medial tibial plateau patch onto the tibia after resection, wherein the MTS medial tibial plateau patch is fixed to the tibia after resection by cement, and the modular MTS medial tibial plateau patch is biologically fixed to the tibia after resection.

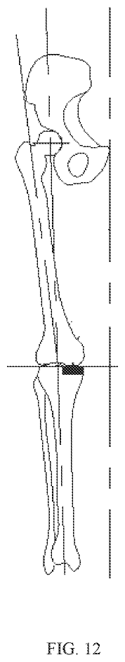

FIG. 11 is an effect diagram of the tibia before replacement, and FIG. 12 is an effect diagram after replacement; by comparing the effect diagrams before and after replacement, when the MTS medial tibial plateau patch or the modular MTS medial tibial plateau patch according to the present invention is implanted onto the tibia with the minimally invasive replacement method according to the present invention, the deformity of the coronal plane within 0-15 degrees may be corrected, with a significant effect.

In the above-mentioned solution, the MTS medial tibial plateau patch and the modular MTS medial tibial plateau patch according to the present invention have simple structures, are convenient to implant and fixed firmly, and have low revision frequency; the surgery is minimally invasive, an incision is small, bone mass and soft tissue may be reserved to the maximum extent, a patient recovers rapidly after the surgery, a current troublesome clinical problem may be solved, better service is provided for the patient, and a market prospect is broad.

Each of the embodiments in the description is described progressively, and each of the embodiments is focused on its differences from other embodiments. The identical or similar portions in each of the embodiments can refer to one another.

In the description of the present invention, it is to be understood that terms such as “upper” and “one end” should be construed to refer to the orientation as then described or as shown in the drawings. These relative terms are for convenience of description and do not indicate or imply that the device or element referred to must have a particular orientation, or be constructed or operated in a particular orientation, and thus cannot be construed to limit the present application.

In the description of the present invention, it should be noted that unless specified or limited otherwise, the terms “provided” and “connected” are used broadly, and may be, for example, fixed connections, detachable connections, or integral connections; may also be direct connections or indirect connections via intervening structures. The fixed connections may be common technical solutions, such as welding, threaded connections, clamping, or the like. The above terms can be understood by those skilled in the art according to specific situations.

It should be noted that the above descriptions are merely preferred embodiments of the present invention, and those skilled in the art may make various improvements and refinements without departing from the principle of the invention. All such modifications and refinements are intended to be covered by the present invention.

What is claimed is:

1. An MTS medial tibial plateau patch, comprising:
an articular surface provided with an arc-shaped recessed area and a bottom surface provided with 3 stand columns distributed triangularly, 2 longitudinal keels having non-parallel distribution or 2 triangular keel wings.

2. The MTS medial tibial plateau patch according to claim 1, wherein each stand column of the 3 stand columns and the bottom surface have an included angle of 20-40 degrees.

3. The MTS medial tibial plateau patch according to claim 1, wherein each stand column of the 3 stand columns is provided with 3-4 arc-shaped grooves, wherein the 3-4 arc-shaped grooves are distributed evenly.

4. The MTS medial tibial plateau patch according to claim 1, wherein each longitudinal keel of the 2 longitudinal keels has a taper of 3-7 degrees.

5. The MTS medial tibial plateau patch according to claim 1, wherein each longitudinal keel of the 2 longitudinal keels is provided with 3-5 convex ridges, wherein the 3-5 convex ridges are distributed uniformly.

6. The MTS medial tibial plateau patch according to claim 1, wherein each triangular keel wing of the 2 triangular keel wings is provided with 2-4 anti-rotation arc grooves.

7. The MTS medial tibial plateau patch according to claim 1, wherein the bottom surface takes a form of one of a surface with a plurality of grid-shaped concave bone cement grooves, a surface with a plurality of dovetail-shaped bone cement grooves and a polished surface without grooves, wherein the plurality of dovetail-shaped bone cement grooves are distributed longitudinally.

8. The MTS medial tibial plateau patch according to claim 1, wherein the MTS medial tibial plateau patch has a D-shaped cross section and is bionically designed according to an anatomical form of a tibial plateau of a knee joint of a Chinese person, and prostheses have models 1-6 with different sizes.

9. The MTS medial tibial plateau patch according to claim 1, further comprising: a plurality of tibial plateau patches with different thicknesses of 3-14 mm.

10. The MTS medial tibial plateau patch according to claim 1, wherein the articular surface has an equal thickness in a front-rear direction.

11. The MTS medial tibial plateau patch according to claim 1, wherein the MTS medial tibial plateau patch is made of VE high cross-linked polyethylene.

12. A modular MTS medial tibial plateau patch, comprising:
a tibial support and a liner, wherein the tibial support comprises an upper surface provided with a conical protrusion and a bottom surface provided with 3-4 stand columns distributed triangularly or 2-3 longitudinal keels having non-parallel distribution; and
the liner comprises an articular surface provided with an arc-shaped recessed area and a lower surface provided with a conical recess; and
the tibial support and the liner are locked by the conical protrusion of the tibial support and the conical recess of the liner.

13. The modular MTS medial tibial plateau patch according to claim 12, wherein the bottom surface of the tibial support is provided with a bone-trabecula porous structure layer with a thickness of 1-5 mm.

14. The modular MTS medial tibial plateau patch according to claim 12, wherein the tibial support is made of medical titanium alloy or cobalt chromium molybdenum alloy or polyether ether ketone (PEEK) or derivatives of the medical titanium alloy or the cobalt chromium molybdenum alloy or the PEEK, and the liner is made of VE high cross-linked polyethylene.

15. A minimally invasive replacement method for replacing an MTS medial tibial plateau patch or a modular MTS medial tibial plateau patch, comprising:
exposing a tibial medial or a lateral plateau of an affected limb of a patient with a minimally invasive surgery method with an incision of a parapatellar approach;
resecting a bone tissue of an affected tibia according to a coronal inversion and eversion angles as well as a sagittal retroversion angle planned before a surgery; and
implanting the MTS medial tibial plateau patch onto a tibia after a resection, wherein an integrated MTS medial tibial plateau patch is fixed to the tibia after the resection by a cement, and the modular MTS medial tibial plateau patch is biologically fixed to the tibia after the resection.
